(19) United States
(12) Patent Application Publication
(10) Pub. No.: US 2026/0199207 A1
(43) Pub. Date: Jul. 16, 2026

(54) COMPOSITION COMPRISING DESFERRIOXAMINES AND A MIXTURE OF TRIGLYCERIDES

(71) Applicant: EVONIK OPERATIONS GMBH, ESSEN (DE)

(72) Inventors: Alexander Thiemann, Hasloh (DE); Marcella Stockstrom, Hamburg (DE); Sarah Alena Strehlow, Bargteheide (DE); Kathrin Daniela Brandt, Duesseldorf (DE)

(73) Assignee: EVONIK OPERATIONS GMBH, ESSEN (DE)

(21) Appl. No.: 19/133,272

(22) PCT Filed: Nov. 22, 2023
(86) PCT No.: PCT/EP2023/082642

§ 371 Date: May 28, 2025

(30) Foreign Application Priority Data
Nov. 30, 2022 (EP) 22210497.8

(51) Int. Cl. A61K 8/37; A61K 8/44; A61Q 19/00

(52) U.S. Cl. CPC A61K 8/37; A61K 8/44; A61Q 19/007

(57) ABSTRACT

The invention relates to desferrioxamines and a mixture of triglycerides.

### FIELD OF THE INVENTION

The invention relates to desferrioxamines and a mixture of triglycerides.

### PRIOR ART

Desferrioxamines form a substance category within the iron-binding siderophores. A review is found, for example, in Hider and Kong, Chemistry and Biology of Siderophores, Natural Product Reports (2010), 27(5), 637-657.

Desferrioxamine B, also known as deferoxamine, is described for application in cosmetic applications. Thus, DE10050155 discloses cosmetic and dermatological preparations with a content of desferrioxamine B.

In recent years, formulations exhibit constantly increasing concentrations of natural oils, in order to satisfy the demand for formulations with certain sensorial properties. Oxidation stable oils, like mineral and silicon oils, thereby become increasingly replaced with natural derived oils. These natural oils are rich in essential unsaturated fatty acids, which depending on the structure, length and degree of saturation, can reveal a wide variety of different sensorial properties.

For instance, natural oils are used as emollients and provide functional and skin sensorial benefits, especially when it comes to the distribution on the skin, one of the most important aspects regarding a pleasant feeling during and after the application.

Furthermore, natural emollients are becoming a preferred alternative as petrochemicals in cosmetics are being increasingly replaced by natural and more sustainable ingredients and in that they do not restrict the respiration of the skin in the same way as synthetic emollients do.

Natural oils, however, often suffer from autoxidation since high levels of unsaturated fatty acids are the target for the oxidative decay.

For solving this problem, formulators use antioxidants and chelating agents to reduce oxidation processes in the formulation.

The use of synthetic antioxidants in cosmetic formulations has been substituted by natural alternatives such as tocopherols, which has become the market benchmark.

However, stabilization of certain natural lipids, especially at higher concentrations, is hard to achieve with tocopherols.

So far no natural derived raw material is capable to overcome this hurdle.

It is an object of the present invention to make available compositions containing a high amount of oils containing unsaturated residues, with enhanced protection from decay caused by oxidation.

### DESCRIPTION OF THE INVENTION

It has been found, surprisingly, that the compositions described subsequently, containing at least one desferrioxamine and/or at least one acid addition product of a desferrioxamine or salts thereof, are able to achieve the object addressed by the invention.

The present invention accordingly relates to compositions containing at least one desferrioxamine and/or at least one acid addition product of a desferrioxamine or salts thereof and triglycerides according to claim 1.

The present invention additionally relates to the use of at least one desferrioxamine and/or at least one acid addition product of a desferrioxamine or salts thereof for the prevention of autoxidation of a mixture comprising at least two triglycerides according to claim 10.

The present invention additionally relates to the use of at least one desferrioxamine and/or at least one acid addition product of a desferrioxamine or salts thereof for decreasing and/or preventing the formation of malodour and/or of toxicological-relevant (e.g. allergens) oxidation degradation products. Such degradation products might be aldehydes, short-chain carboxylic acids, ketones and mono- and polycyclic hydrocarbons (PAHs), compare for example Coupland et al. Trends Food Sci Technol. 1996; 7(3): 83-91. These oxidation degradation products have, among other things, the potential to increase the allergenic potential when applied regularly to the skin, compare for example De Rentiis et al. Conatct Deramtitis. 2021; 84(5): 317-325; Huang et al. Tenside Surfactants Detergents. 2020; 7(3): 230-236; Damevska et al. In: Advances in Integrative Dermatology. John Wiley Blackwell, USA, 2019.; O'Brien et al. Crit Rev Toxicol. 2005; 35(7): 609-662.

Toxicological-relevant oxidation degradation products (e.g. allergens) the use of the instant invention preferably is preventing the formation of are selected from but not limited to the group of primary oxidation products such as peroxides and hydroperoxides (Christensson et al. Contact Dermatitis. 2012 67(5): 247-59) monoterpenes (e.g. 7-hydroperoxy-3,7-dimethylocta-1,5-diene-3-ol), monoterpenoid-esters, lipids (e.g. 9-hydroperoxy octadecadienoic acid, 13-hydroperoxy octadecadienoic acid), and secondary oxidation products which might be toxicological-relevant themselves or indirect via reaction with other components such as proteins. Such secondary toxicological-relevant oxidation products are preferably selected from alcohols, aldehydes, ketones, organic acids, esters, amides, amines, alkenes, cyclic alkenes, cyclic heteroalkanes, cyclic heteroalkenes, atromatic compounds, heteroaromatic compounds.

Especially the oxidation products of cholesterol (e.g. 7alpha-hydroxycholesterol, 7β-hydroxycampesterol, 5α-cholestan-3β,5α,6β triol), Linalool (e.g. 2-(5-methyl-5-vinyltetrahydrofuran-2-yl)propan-2-ol, 2,2,6-trimethyl-6-vinyltetrahydro-2H-pyran-3-ol, 2,6-dimethylocta-3,7-diene-2,6-diol, 2,6-dimethylocta-1, 7-diene-3,6-diol) and Phytosterols (e.g. 7α-hydroxysitosterol, 7β-hydroxysitosterol, sitosterol-5α,6α-epoxide, sitosterol-5β,6β-epoxide, 7α-hydroxycampeterol, 7β-hydroxycampesterol, sitostanetriol, 7-ketocampesterol, 7-ketositosterol), aldehydes such as acetaldehyde, dienals, acrolein, 4-hydroxnonenal and low-molecular-weight aldehydes with one to eight carbon atoms and organic acids such as acetic acid and formic acid, are preferably decreasing and/or their formation is prevented by the use according to the instant invention.

An advantage of the present invention is that the compositions according to the invention have good spreadability and thus achieve a homogeneous distribution and pleasant feeling on the skin during and after the application.

An additional advantage of the present invention is that the compositions according to the invention exhibit particularly good skin sensorics and contribute to a reduction in the tackiness of the formulation.

An additional advantage of the present invention is that the compositions according to the invention are characterized by a relatively low oily and satisfactorily absorbing feeling on the skin.

An additional advantage of the present invention is that the compositions according to the invention exhibit an excellent shelf life with regard to the emulsion stability and accordingly can be kept for a long time.

An additional advantage of the present invention is that the compositions according to the invention have a particularly insignificant influence on the viscosity of a formulation and can be used over a broad pH range.

An additional advantage of the present invention is that the compositions according to the invention can be incorporated in a particularly easy and stable manner in cosmetic formulations.

An additional advantage of the present invention is that the compositions according to the invention contribute to an improvement in the thermal stability of the formulation.

An additional advantage of the present invention is that the compositions according to the invention impart an excellent colour stability in the formulation.

An additional advantage of the present invention is that the compositions according to the invention exhibit improved shelf lives with regard to growth of certain microorganisms and accordingly can be kept for a long time.

The present invention accordingly relates to a composition comprising
A) at least one substance chosen from desferrioxamines and acid addition products of a desferrioxamine or salts thereof,
B) a mixture comprising at least two triglycerides,
wherein the mixture of the at least two triglycerides comprised in the composition has an iodine value of from 6 cg I/g to 300 cg I/g, preferably from 30 cg I/g to 270 cg I/g, more preferably from 50 cg I/g to 250 cg I/g, most preferably from 70 cg I/g to 240 cg I/g,
characterized in that component B) is present in an amount of 5.0% by weight to 80% by weight, preferably from 8.0% by weight to 60% by weight, particularly preferably from 10.0% by weight to 30% by weight, the percentages by weight being with respect to the total composition.

The iodine value (IV) is a measure of the total number of double bonds present in for example fats and oils. It is expressed as the number of grams of iodine that will react with the double bonds in 100 grams of substrate (for example fats or oils) or as centigram iodine per gram of substrate. The iodine value is preferably determined by the Wijs/Hanuš method, preferably by using applied thermometric titration, for example as described in Thermo. Titr. Application Note No. H-076 published by Metrohm UK Ltd.

The iodine value in context with the instant invention refers to the mixture of all triglycerides comprised in the composition according to the instant invention in total.

Unless otherwise indicated, all stated percentages (%) are percentages by weight.

Any desferrioxamine and acid addition product of a desferrioxamine or salt thereof, and also mixtures thereof, can be present according to the invention in component A).

Preferred desferrioxamines in this connection are chosen from desferrioxamine A_{1A}, desferrioxamine A_{1B}, desferrioxamine A₂, desferrioxamine B, desferrioxamine D₁, desferrioxamine D₂, desferrioxamine E, desferrioxamine Et₁, desferrioxamine Et₂, desferrioxamine Et₃, desferrioxamine G₁, desferrioxamine G_{2A}, desferrioxamine G_{2B}, desferrioxamine G_{2C}, desferrioxamine H, desferrioxamine N, desferrioxamine P₁, desferrioxamine T₁, desferrioxamine T₂, desferrioxamine T₃, desferrioxamine T₇, desferrioxamine T₈, desferrioxamine Te₁, desferrioxamine Te₂, desferrioxamine Te₃, desferrioxamine X₁, desferrioxamine X₂, desferrioxamine X₃ and desferrioxamine X₄, preferably desferrioxamine B, desferrioxamine D₁, desferrioxamine D₂, desferrioxamine E and desferrioxamine H, particularly preferably desferrioxamine B and desferrioxamine E.

For the nomenclature, compare if necessary Hider and Kong, Chemistry and Biology of Siderophores, Natural Product Reports (2010), 27(5), 637-657.

It is preferred according to the invention for the acid addition product of the desferrioxamine or salts thereof to be chosen from acid addition products of the acids chosen from formic acid, aminooxyacetic acid, amino acids, anthraquinonecarboxylic acid, succinic acid, aqueous hydrochloric acid, acetic acid, folic acid, glutaric acid, hydroxamic acid, malonic acid, methanesulfonic acid, nalidixic acid, N-(methylamino)benzoic acid, phenylsuccinic acid, phenylglutaric acid, phosphorous acid, phosphoric acid, hydrogen chloride, sulfurous acid, sulfuric acid, tartaric acid and citric acid, hydrogen chloride, methanesulfonic acid, acetic acid and amino acids being particularly preferred.

A preferred composition according to the invention is characterized in that component A) is present in an amount of 0.005% by weight to 10.0% by weight, preferably from 0.05% by weight to 5.0% by weight, particularly preferably from 0.1% by weight to 1.0% by weight, the percentages by weight being with respect to the total composition.

When determining the amount of component A) only the mass of the desferrioxamine is taken into account while disregarding the counter ion of the salt or the part of the acid in the acid addition product, if the case.

A preferred composition according to the invention is characterized in that the mixture comprised in component B) comprises at least three, preferably at least four, particularly preferably at least five, triglycerides.

A preferred composition according to the invention is characterized in that the mixture comprised in component B) is obtained from natural sources.

A preferred composition according to the invention is characterized in that the mixture comprised in component B) is selected from natural oils, natural waxes, natural butters, essential oils and mixtures thereof.

It is preferred according to the invention for component B) of the composition according to the invention to comprise at least one, preferably at least two, particularly preferably at least three, natural oils.

Preferred natural oils comprised in component B) in the composition of the instant invention are selected from the group of Abyssinia oil (Crambe abyssinica oil), Abyssinia seed oil (Crambe abyssinica seed oil), Acai berry oil, Acai fruit oil (Euterpe Oleracea (fruit) oil), Andiroba oil, Apricot kernel oil (Prunus Armeniaca Kernel Oil), Argan oil, Avocado oil (Persea gratissima oil), Almond oil, Amaranth oil, Babassu seed oil (Orbignya Oleifera (Seed) Oil), Baobab oil (Adansonia digitata oil), Baobab seed oil (Adansonia digitata Seed Oil), Blackberry seed oil, Canola oil, Carrot macerated oil, Castor oil (Ricinus communis seed oil), Corn oil (Zea mays oil), Cranberry seed oil, Evening primrose oil (Oenothera biennis oil), Cottonseed oil (Gossypium herbaceum seed oil), Glycine soja oil, Goji seed oil, Guava seed oil, Grape seed oil (Vitis vinifera seed oil), Hemp seed Oil (Canabis sativa seed oil), Hybrid sunflower oil (Helianthus annuus hybrid oil), Lineseed oil (Linum usitatissimus oil), Lineseed seed oil (Linum usitatissimus seed oil), Macadamia nut oil, Macadamia ternifolia seed oil, Macadamia integrifolia seed oil, Maracuja oil (Passiflora edulis oil), Marula seed oil (Sclerocarya birrea seed oil), Meadowfoam seed oil (Limnanthes alba seed oil), Oat oil, Squalane, Olus oil, Jojoba oil (Simmondsia chinensis seed oil), Rice bran oil (Oryza sativa bran oil), Sunflower oil (Helianthus annuus (sunflower) seed oil), Sweet almond oil, Olive oil (Olea europaea (olive) fruit oil), Palm oil (Elaeis guineensis oil), Palm kernel oil, Paranussöl (Bertholletia excelsa seed oil), Passionfruit seed oil (Passiflora edulis seed oil), Peach kernel oil, Peel oil, Pequi oil, Pomegranate seed oil (Punica granatum seed oil), Prickly Pear seed oil, Pumpkin seed oil, Raspberry seed oil (Rubus idaeus seed oil), Rose oil (Rosa damascena oil), Rose flower oil (Rosa damascena flower oil), Rosehip seed oil, Coconut oil (Cocos nucifera (coconut) oil, Hazelnut oil, Safflower oil, Safflower seed oil (Carthamus tinctorius seed oil), Seed oil, Sesame (seed) oil (Sesamum indicum (seed) oil), Tea tree leaf oil (Melaleuca alternifolia Leaf Oil), Walnut oil and Wheat germ oil,
with the group of Helianthus annuus (seed) oil, Coconut oil (Cocos nucifera oil), Jojoba (seed) oil (Simmondsia chinensis (seed) oil), Sweet almond oil (Prunus amygdalus dulcis oil), Castor oil, Olive (fruit) oil (Olea europaea (fruit) oil), Argan (kernel) oil (Argana spinosa (kernel) oil), Avocado oil (Persea gratissima oil), Soybean oil (Glycine soja oil), Palm oil, Palm kernel oil, Grape (seed) oil (Vitis vinifera (seed) oil), Lavender essential oil (Lavandula angustifolia oil), Macadamia nut (seed) oil (Macadamia ternifolia (seed) oil), Tea tree (leaf) oil (Melaleuca alternifolia (leaf) oil), Apricot (kernel) oil (Prunus armeniaca (kernel) oil), Rosehip (fruit) oil (Rosa canina (fruit) oil), Safflower (seed) oil (Carthamus tinctorius (seed) oil), Rosemary (leaf) oil (Rosmarinus officinalis (leaf) oil), Lemon (peel) oil (Citrus medica limonum (peel) oil), Bergamot (fruit) oil (Citrus aurantium bergamia fruit oil), Meadowfoam (seed) oil (Limnanthes alba (seed) oil), Eucalyptus (leaf) oil (Eucalyptus globulus (leaf) oil), Olus oil and Hemp (seed) oil (Cannabis sativa (seed) oil) being particularly preferred.

Preferred natural waxes in component B) in the composition of the instant invention are selected from the group of Carnauba (Copernicia cerifera (carnauba) wax), Candelilla (Euphorbia cerifera (candelilla) wax), Beeswax (beeswax) and Sunflower wax (Helianthus annuus (sunflower) seed wax), Hemp wax, Almond wax, Myrica fruit wax, Rice bran wax, Cananga odorata flower cera (Ylang Ylang Flower Wax), Jasminum grandiflorum flower cera (Jasmine Flower Wax), Lavandula angustifolia flower cera (Lavender Flower Wax), Rosa damascene flower cera (Rose Flower Wax) and Acacia decurrens flower cera (Mimosa Flower Wax).

Preferred natural butters in component B) in the composition of the instant invention are selected from the group of Astrocaryum Murumuru Seed Butter, Astrocaryum vulgare seed butter, Cocoa butter (Theobroma cacao seed butter), Shea butter (Butyrospermum parkii butter), Theobroma Grandiflorum Seed Butter, Virola surinamensis Seed Butter, Platonia insignis Seed Butter, Shorea Stenoptera Seed Butter, Garcinia indica Seed Butter, Mangifera indica Seed Butter and Virola sebifera nut butter

Preferred essential oils in component B) in the composition of the instant invention are selected from the group of: Tea Tree oil (Melaleuca alternifolia (tea tree) leaf oil), Copaiba oil, Agar oil (Aquilaria malaccensis oil), Anise oil (Pimpinella anisum oil), Basil oil, Bergamot oil, Bergamot fruit oil (Citrus aurantium bergamia fruit oil), Birch oil, Borage seed oil (Borago officinalis seed oil), Camellia seed oil (Camellia japonica seed oil), Camellia oleifera (seed) oil, Camphor oil, Cannabis flower essential oil, Cedar oil, Chamomile flower oil (Anthemis nobilis flower oil), Cinnamon oil, Citron oil, Citronella oil, Coconut oil, Eucalyptus oil, Eucalyptus leaf oil (Eucalyptus globulus leaf oil), Fennel seed oil, Garlic oil, Geranium oil (Perlagonium graveolens oil), Geranium flower oil (Pelargonium graveolens flower oil), Ginger oil, Grapefruit oil (Citrus grandis oil), Grapefruit peel oil (Citrus grandis peel oil), Hickory nut oil, Horseradish oil, Jasmine oil, Juniper berry oil, Lavender oil (Lavandula angustifolia oil), Lavandin oil (Lavandula hybrida oil), Lavandula officinalis flower oil, Lemon oil, Lemon peel oil (Citrus medica limonum peel oil), Mandarin orange peel oil (Citrus nobilis peel oil), Melissa oil, Moringa oil (Moringa oleifera oil), Moringa seed oil (Moringa oleifera seed oil), Mustard oil, Neem oil, Orange oil (Citrus aurantium dulcis oil), Orange peel oil (Citrus aurantium dulcis peel oil), Oregano oil, Patchouli oil (Pogostemon cabin oil), Peppermint oil (Mentha piperita oil), Peppermint herb oil (Mentha piperita herb oil), Pine oil, Rose oil, Roshehip oil, Rosehip fruit oil (Rosa canina fruit oil), Rosemary oil, Rosemary leaf oil (Rosmarinus officinalis leaf oil), Rosewood oil, Sage oil, Sandalwood oil, Sassafras oil, Savory oil, Star anise oil, Tangerine, Thyme oil, Wheat germ oil (Triticum vulgare germ oil), Wintergreen oil, Wild mint leaf oil (Mentha arvensis leaf oil), Yarrow oil and Ylang Ylang flower oil (Cananga odorata flower oil).

The compositions according to the invention can, e.g., comprise at least one further additional component chosen from the group of the
emollients, emulsifiers, coemulsifiers, thickeners/viscosity regulators/stabilizers, antioxidants, hydrotropes (or polyols), solids and fillers, pearlescent additives, insect repellents, self-tanning agents, preservatives, conditioners, perfumes, colourants, cosmetic active substances, care additives, superfatting agents and solvents.

From the term “further additional component” it is obvious, that component A) and B) comprised in the composition of the instant invention are different from said additional components and that component A) and B) are not accounted to said additional components.

Substances which can be used as exemplary representatives of the individual groups are known to a person skilled in the art and can, for example, be taken from German Application DE 102008001788.4. This patent application is incorporated in the current document as reference and is accordingly to be regarded as part of the disclosure.

As regards further optional components and also the amounts used of these components, reference is expressly made to the relevant handbooks known to a person skilled in the art, e.g. K. Schrader, “Grundlagen und Rezepturen der Kosmetika [Fundamentals and Formulations of Cosmetics]”, 2nd edition, pages 329 to 341, Hüthig Buch Verlag, Heidelberg.

The amounts of the respective additives depend on the intended use.

Typical starting formulations for the relevant applications are known prior art and are contained for example in the brochures of the manufacturers of the relevant base materials and active substances. These existing formulations can generally be adopted unchanged. However, if necessary, for adjustment and optimization, the desired modifications can be executed in a straightforward manner through simple tests.

Compositions according to the invention can, for example, be used as skin care, face care, head care, body care, intimate care, foot care, hair care, nail care, dental care or oral care products.

Compositions according to the invention can, for example, be used in the form of an emulsion, a suspension, a solution, a cream, a salve, a paste, a gel, a stick, a spray, a cleansing product, a make-up or sunscreen preparation or a face lotion.

Preferred compositions according to the invention are emulsions, in particular chosen from oil-in-water emulsions and water-in-oil emulsions, oil-in-water emulsions being particularly preferred.

The present invention additionally relates to the use of at least one substance chosen from desferrioxamines and acid addition products of a desferrioxamine or salts thereof, in particular desferrioxamine B, for the prevention of autoxidation of a mixture comprising at least two triglycerides, wherein the mixture of the at least two triglycerides comprised in the composition has an iodine value of from 6 cg I/g to 300 cg I/g, preferably from 30 cg I/g to 270 cg I/g, more preferably from 50 cg I/g to 250 cg I/g, most preferably from 70 cg I/g to 240 cg I/g.

The present invention additionally relates to the use of at least one desferrioxamine and/or at least one acid addition product of a desferrioxamine or salts thereof, in particular desferrioxamine B, for decreasing and/or preventing the formation of malodour of a composition, preferably of a mixture comprising at least two triglycerides, wherein the mixture of the at least two triglycerides comprised in the composition has an iodine value of from 6 cg I/g to 300 cg I/g, preferably from 30 cg I/g to 270 cg I/g, more preferably from 50 cg I/g to 250 cg I/g, most preferably from 70 cg I/g to 240 cg I/g.

The present invention additionally relates to the use of at least one desferrioxamine and/or at least one acid addition product of a desferrioxamine or salts thereof, in particular desferrioxamine B, for decreasing and/or preventing the formation of toxicological-relevant oxidation degradation products in a composition, preferably in a mixture comprising at least two triglycerides, wherein the mixture of the at least two triglycerides comprised in the composition has an iodine value of from 6 cg I/g to 300 cg I/g, preferably from 30 cg I/g to 270 cg I/g, more preferably from 50 cg I/g to 250 cg I/g, most preferably from 70 cg I/g to 240 cg I/g.

In the uses according to the invention, the components mentioned above in connection with the compositions according to the invention as components preferably present are preferably used.

The present invention is described by way of example in the examples cited below, without the invention, the scope of application of which results from the entirety of the description and from the claims, being restricted to the embodiments mentioned in the examples.

### EXAMPLES

### Example 1

For the comparison of the ability to combat oxidation processes in compositions comprising high amounts of natural, non-hydrogenated oils, different cosmetic compositions (Formulations 1-4) were prepared.

Oxidation stability was measured via oxygen levels in headspace over a formulation inside a closed system.

The formulation constituents in the following compositions (Formulations 1-6) are referred to in the form of the generally accepted INCI nomenclature with use of the English terms. All concentrations in the use examples are, unless otherwise indicated, given as % by weight.

### Formulations 1-4: O/W Emulsions

In total, 4 O/W emulsions were prepared.

Formulation 1 comprised no antioxidant or chelating agent (neg. control) and Formulations 2-4 contained either desferrioxamine, Disodium EDTA or Tocopherols. The pH of all formulations was adjusted with citric acid to 5,5.

| | comp. | * Formulation | comp. | comp. |
|---|---|---|---|---|
| Raw material (INCI EU) | Formulation 1 | 2 | Formulation 3 | Formulation 4 |
| Deionised Water | 68.6 | 68.42 | 68.5 | 68.1 |
| Glycerin | 5.00 | 5.00 | 5.00 | 5.00 |
| Xanthan Gum | 0.3 | 0.3 | 0.3 | 0.3 |
| Desferrioxamin B | — | 0.18 | — | — |
| EDTA (disodium) | — | — | 0.10 | — |
| Glyceryl Stearate Citrate | 3.50 | 3.50 | 3.50 | 3.50 |
| Caprylic/Capric Triglyceride | 2.00 | 2.00 | 2.00 | 2.00 |
| Helianthus Annuus Seed Oil | 15.00 | 15.00 | 15.00 | 15.00 |
| Cetearyl Alcohol | 2.5 | 2.5 | 2.5 | 2.5 |
| Tocopherol | — | — | — | 0.5 |
| Methylpropanediol, Caprylyl Glycol, | 3.0 | 3.0 | 3.0 | 3.0 |
| Phenylpropanol, Ethylhexyl | | | | |
| Salicylate | | | | |
| Iron Chloride | 0.1 | 0.1 | 0.1 | 0.1 |

The results (Table 1) show that, with Formulations 2 according to the invention (*) containing desferrioxamine, in comparison to comparative Formulation 1, more efficient oxidation protection is surprisingly achieved and that, surprisingly, the effectiveness turned out to be higher than the effectiveness through the functionally similar comparative substance Disodium EDTA (comparative Formulation 3) and the benchmark antioxidant Tocopherol (comparative Formulation 4).

TABLE 1
Oxidation stability results (average values)

| | comp. | * | comp. | comp. |
|---|---|---|---|---|
| | Formulation | Formulation | Formulation | Formulation |
| | 1 | 2 | 3 | 4 |
| Oxidation | 1.00 | 3.75 | 1.37 | 2.19 |
| stability value | | | | |
| (rel.) | | | | |

### Example 2

Sensory improvements of a formulation according to the invention (Formulation 2 from Example 1) in comparison to a formulation without desferrioxamine (comp. Formulation 1 from Example 1) and a formulation with tocopherols (comp Formulation 4 from Example 1) and one formulation with Disodium EDTA (comp Formulation 3 from Example 1) are observed.

The malodour-formation was tested by observing the rancid smell of each formulation in comparison to rancid-smelling-standard-control on a 7-point scale. The differences between Formulation 1-4 were evaluated from 0 (no difference) to 6 (very big difference) by a group of 5 trained test persons after a storage period (4 months, 45° C.).

Spreadability attributes of the formulations were assessed on the internal side of the forearm. Tests were carried out successively on the left and right arms.

The formulations were evaluated by a panel of 5 assessors. Emulsions were evaluated for spreadability (appropriate 5-point scales were structured) represented as the area that the emulsion will cover while being rubbed 8 times in a circular motion on the forearm (defined 4-cm-diameter circle)—0: no spreadability (Emulsion completely visible, strong whitening effect); 1: slight spreadability (Emulsion visible, slight whitening effect); 2: reliable spreadability (Emulsion less visible, less whitening effect); 3 good spreadability (Emulsion not visible, no whitening effect only oily shine); 4: very good spreadability (Emulsion not visible, no whitening effect, no oily shine)

The sensory test results are summarized in Table 2.

TABLE 2
Sensory rating (average values)

| | comp. | * | comp. | comp. |
|---|---|---|---|---|
| | Formulation | Formulation | Formulation | Formulation |
| | 1 | 2 | 3 | 4 |
| Rancid smell - | 1.6 | 5 | 3.8 | 4.4 |
| differences | | | | |
| Spreadability | 3.0 | 3.8 | 3.0 | 3.0 |

The emulsion formulation according to the invention (Formulation 2), surprisingly improves the spreadability and decreases the malodour formation in comparison to the vehicle formulation (comp Formulation 1) without desferrioxamine and in comparison to formulations with Tocopherols (comp Formulation 4) and Disodium EDTA (comp Formulation 3).

### Example 3

Further sensory improvements of a formulation according to the invention (Formulation 2 from Example 1) in comparison to a formulation without desferrioxamine (comp. Formulation 1 from Example 1) and a formulation with tocopherols (comp Formulation 4 from Example 1) and one formulation with Disodium EDTA (comp Formulation 3 from Example 1) are observed.

Sensory attributes of the formulations were assessed on the internal side of the forearm. Tests were carried out successively on the left and right arms.

The formulations were evaluated by a panel of 5 assessors. Emulsions were evaluated for tackiness and oily skin feeling (appropriate 5-point scales were structured) after the emulsion being rubbed 8 times in a circular motion on the forearm—0: low; 1: slight; 2: medium; 3 high; 4: very high).

The sensory test results are summarized in Table 3.

TABLE 3
Sensory rating (average values)

| | comp. | * | comp. | comp. |
|---|---|---|---|---|
| | Formulation | Formulation | Formulation | Formulation |
| | 1 | 2 | 3 | 4 |
| Tackiness | 2 | 1.6 | 1.8 | 1.8 |
| Oily skin | 2.2 | 1.2 | 1.6 | 2.0 |
| feeling | | | | |

The emulsion formulation according to the invention (Formulation 2), surprisingly reduced the tackiness and oily skin feeling in comparison to the vehicle formulation (comp Formulation 1) without desferrioxamine and in comparison to formulations with Tocopherols (comp Formulation 4) and Disodium EDTA (comp Formulation 3).

### Example 4

Stability improvements of a formulation according to the invention (Formulation 2 from Example 1) in comparison to a formulation without desferrioxamine (comp. Formulation 1 from Example 1) are observed.

Stability was assessed by viscosity measurement (DV Next, Ametek Brookfield) of the formulation after an incubation period of 1 year at RT (in darkness).

The stability test results are summarized in Table 4.

TABLE 4
Viscosity data

| | Formulation 1 (without | Formulation 2 (with |
|---|---|---|
| | desferrioxamine) | desferrioxamine) |
| Viscosity (mPas; speed | 48.800 | 51.300 |
| 10 rpm) | | |

The formulation according to the invention (Formulation 2), surprisingly showed a higher viscosity and therefore as an emulsion a higher stability, in comparison to the vehicle formulation (comp Formulation 1) without desferrioxamine after a storage period of 1 year at RT.

### Example Formulations

The formulation constituents in the following compositions (Formulations 1-4) are referred to in the form of the generally accepted INCI nomenclature with use of the English terms. All concentrations in the use examples are, unless otherwise indicated, given as % by weight.

### Natural Handcream

| | conc. % by |
|---|---|
| INCI (EU) | weight |
| Aqua | 41.40 |
| Glycerin | 3.00 |
| Desferrioxamine | 0.10 |
| Xanthan Gum | 0.50 |
| Glyceryl Stearate Citrate; Cetearyl Alcohol; Glyceryl Caprylate | 6.00 |
| Oryza Sativa Cera | 3.00 |
| Theobroma Grandiflorum Seed Butter | 2.00 |
| Helianthus Annuus Seed Oil | 23.00 |
| Decyl Cocoate | 5.00 |
| Olea Europaea Fruit Oil | 5.00 |
| Persea Gratissima Oil | 2.00 |
| Tocopherol; Helianthus Annuus Seed Oil | 0.20 |
| Aqua; Sodium Lauroyl Lactylate; Ceramide NP; Phenoxyethanol; Ceramide AP; | 5.00 |
| Phytosphingosine; Cholesterol; Xanthan Gum; Carbomer; Ethylhexylglycerin; | |
| Ceramide EOP | |
| Glycerin; Aqua; Sodium Levulinate; Sodium Anisate | 3.50 |
| Parfum | 0.30 |

### Natural Body Lotion

| | conc. % by |
|---|---|
| INCI (EU) | weight |
| Aqua | 65.85 |
| Glycerin | 3.00 |
| Desferrioxamine | 0.30 |
| Xanthan Gum | 0.30 |
| Sclerotium Gum | 0.15 |
| Polyglyceryl-3 Distearate; Glyceryl Stearate Citrate | 3.00 |
| Cetearyl Alcohol | 1.00 |
| Helianthus Annuus Seed Cera; Tocopherol; Ascorbyl Palmitate | 2.00 |
| Butyrospermum Parkii Butter | 2.00 |
| Helianthus Annuus Seed Oil | 6.00 |
| Carthamus Tinctorius Seed Oil | 5.00 |
| Isoamyl Laurate | 1.00 |
| Daucus Carota Sativa Root Extract; Sesamum Indicum Seed Oil; Rosmarinus | 3.00 |
| Officinalis Leaf Extract; Helianthus Annuus Seed Oil | |
| Tocopherol; Helianthus Annuus Seed Oil | 0.20 |
| Glyceryl Caprylate | 0.30 |
| Aqua; Sodium Levulinate; Potassium Sorbate | 1.50 |
| Aqua; Sodium Lauroyl Lactylate; Ceramide NP; Phenoxyethanol; Ceramide AP; | 5.00 |
| Phytosphingosine; Cholesterol; Xanthan Gum; Carbomer; Ethylhexylglycerin; | |
| Ceramide EOP | |
| Parfum | 0.40 |

### Natural Oil Release Cream

| | conc. % |
|---|---|
| INCI (EU) | by weight |
| Polyglyceryl-6 Stearate (and) Polyglyceryl-6 Behenate) | 3.00 |
| Glyceryl Stearate | 1.00 |
| Cetearyl Alcohol | 1.00 |
| Decyl Cocoate | 35.00 |
| Helianthus Annuus (Sunflower) Seed Oil | 34.60 |
| Glyceryl Caprylate | 0.40 |
| Water | 20.40 |
| Glycerin | 2.00 |
| Cellulose | 2.00 |
| Desferrioxamine | 0.4 |
| Sodium Anisate | 0.20 |
| 10% sol. In water | q.s. |
| Parfum | q.s. |

### Natural Body Lotion

| | conc. |
|---|---|
| | % by |
| INCI (EU) | weight |
| Aqua | 72.10 |
| Desferrioxamine | 0.10 |
| Glycerin | 4.50 |
| Sodium Anisate | 0.30 |
| Xanthan Gum | 0.20 |
| Distarch Phosphate | 1.00 |
| Carrageenan | 0.10 |
| Glyceryl Stearate Citrate; Cetearyl Alcohol; Glyceryl Caprylate | 4.00 |
| Simmondsia Chinensis Seed Oil | 2.00 |
| Isoamyl Laurate | 2.00 |
| Helianthus Annuus Seed Oil | 10.00 |
| Astrocaryum Murumuru Seed Butter | 1.00 |
| Tocopherol; Helianthus Annuus Seed Oil | 0.50 |
| Levulinic Acid; Sodium Levulinate; Aqua; Glycerin | 0.70 |
| Parfum | 0.50 |
| Aqua; Cyanidium Caldarium Extract | 1.00 |

### Natural Baby Cream

| | conc. % |
|---|---|
| INCI (EU) | by weight |
| Aqua | 63.35 |
| Glycerin | 5.00 |
| Desferrioxamine | 0.50 |
| Zinc Sulfate | 1.00 |
| Polyglyceryl-4 Diisostearate/Polyhydroxystearate/Sebacate | 6.00 |
| Glyceryl Caprate | 0.30 |
| Prunus Amygdalus Dulcis Oil | 8.00 |
| Sesamum Indicum Seed Oil | 4.00 |
| Cera Alba | 0.40 |
| Decyl Cocoate | 6.00 |
| Olus Oil | 5.00 |
| Tocopherol; Helianthus Annuus Seed Oil | 0.20 |
| Parfum | 0.25 |

### Natural Care Deo Stick

| | conc. % |
|---|---|
| INCI (EU) | by weight |
| Myristyl Myristate | 2.00 |
| Helianthus Annuus Seed Oil | 10.00 |
| Isoamyl Laurate | 5.00 |
| Decyl Cocoate | 11.50 |
| Triethyl Citrate | 5.00 |
| Cetearyl Alcohol | 4.00 |
| Polyglyceryl-2 Sesquioleate | 1.00 |
| Olus Oil | 9.00 |
| Zinc Ricinoleate | 1.50 |
| Hydrogenated Vegetable Oil | 6.00 |
| Polyglyceryl-3 Caprylate | 0.50 |
| Theobroma Grandiflorum Seed Butter | 3.00 |
| Candelilla Cera | 6.00 |
| Copernicia Cerifera Cera | 3.00 |
| Cera Alba | 7.00 |
| Sodium Caproyl/Lauroyl Lactylate; Triethyl | 0.50 |
| Citrate; Salvia Officinalis Oil | |
| Tocopherol; Helianthus Annuus Seed Oil | 0.50 |
| Cellulose | 15.00 |
| Glyceryl Caprylate | 0.50 |
| Zinc Oxide | 3.00 |
| Aqua | 4.90 |
| Desferrioxamine | 0.10 |
| Parfum | 1.00 |

### Natural Green Body Mask

| | conc. % |
|---|---|
| INCI (EU) | by weight |
| Aqua | 57.20 |
| Glycerin | 4.50 |
| Desferrioxamine | 0.20 |
| Sodium Phytate; Aqua; Alcohol | 0.10 |
| Xanthan Gum | 0.60 |
| Squalane | 5.00 |
| Phytosphingosine | 0.20 |
| Glyceryl Stearate Citrate; Cetearyl Alcohol; | 8.00 |
| Glyceryl Caprylate | |
| Glyceryl Stearate | 2.00 |
| Cera Alba | 2.00 |
| Camellia Sinensis Leaf Extract | 2.00 |
| Butyrospermum Parkii Butter | 2.00 |
| Prunus Amygdalus Dulcis Oil | 5.00 |
| Citrus Aurantium Bergamia Fruit Oil | 3.00 |
| Prunus Armeniaca Kernel Oil | 2.00 |
| Tocopherol; Helianthus Annuus Seed Oil | 0.20 |
| Tocopherol; Helianthus Annuus Seed Oil | 1.00 |
| Curcuma Longa Root Extract | 0.50 |
| Helianthus Annuus Hybrid Oil; Cucurbita Pepo Seed | 0.50 |
| Extract; Rosmarinus Officinalis Leaf Extract | |
| Aqua; Cyanidium Caldarium Extract | 0.50 |
| Glycerin; Aqua; Sodium Levulinate; Sodium Anisate | 3.50 |

### Moisturizing and Caring Serum

| | conc. % |
|---|---|
| INCI (EU) | by weight |
| Glycerin | 3.00 |
| Aqua | 74.00 |
| Desferrioxamine | 0.10 |
| Hydrolyzed Vegetable Protein; Sodium Citrate; | 2.00 |
| Magnesium Stearate; Xanthan Gum | |
| Glyceryl Stearate | 0.50 |
| Glyceryl Stearate Citrate | 0.20 |
| Isoamyl Laurate | 2.00 |
| Simmondsia Chinensis Seed Oil | 4.00 |
| Mangifera Indica Seed Butter | 0.50 |
| Tocopherol; Helianthus Annuus Seed Oil | 0.20 |
| Prunus Amygdalus Dulcis Oil | 4.00 |
| Pentylene Glycol | 4.00 |
| Aqua; Alcohol; Glycosphingolipids; Hordeum Vulgare | 5.00 |
| Extract; Sodium Hyaluronate | |
| Parfum | 0.50 |

### W/O Cream

| | conc. % |
|---|---|
| INCI (EU) | by weight |
| Polyglyceryl-4 Diisostearate/Polyhydroxystearate/ | 3.00 |
| Sebacate; Caprylic/Capric Triglyceride; Polyglyceryl-3 | |
| Oleate, Diisostearoyl Polyglyceryl-3 Dimer Dilinoleate) | |
| Caprylic/Capric Triglyceride | 10.00 |
| Squalane | 8.00 |
| Helianthus Annuus (sunflower) Seed Oil | 8.00 |
| KahlWax 8104 (Cera Alba) | 4.00 |
| Glyceryl Caprate | 0.50 |
| Tocopherol; Helianthus Annus (Sunflower) Seed Oil) | 0.20 |
| Water | 60.10 |
| Glycerin | 5.00 |
| Desferrioxamine | 0.20 |
| Zinc Sulfate Heptahydrate | 1.50 |
| Perfume | q.s. |

### Everyday Soft Cream

| | conc. % |
|---|---|
| INCI (EU) | by weight |
| Polyglyceryl-3 Distearate; Glyceryl Stearate Citrate) | 3.00 |
| Glyceryl Stearate | 2.00 |
| Stearyl Alcohol | 1.00 |
| Helianthus Annuus (Sunflower) Seed Oil | 11.00 |
| Rosmarinus Officinalis Leaf Oil | 2.00 |
| Eucaplyptus Globolus Leaf Oil | 2.00 |
| Tocopherol | 0.10 |
| Water | 72.00 |
| Glycerin | 3.00 |
| Desferrioxamine | 0.20 |
| Keltrol CG-SFT (Xanthan Gum) | 0.20 |
| Genuvisco carrageenan CG-131 (Carrageenan) | 0.20 |
| Glycerin; Aqua; Sodium Levulinate; Sodium Anisate | 3.50 |

### Natural Buttercream

| | conc. % |
|---|---|
| INCI (EU) | by weight |
| Aqua | 35.00 |
| Glycerin | 3.00 |
| Desferrioxamine | 0.2 |
| p-Anisic Acid | 0.10 |
| Glyceryl Stearate Citrate; Cetearyl Alcohol; | 7.00 |
| Glyceryl Caprylate | |
| Decyl Cocoate | 18.00 |
| Cannabis Sativa Seed Oil | 1.00 |
| Tocopherol; Helianthus Annuus Seed Oil | 0.50 |
| Helianthus Annuus Seed Oil | 35.00 |
| Parfum | 0.20 |

### Natural Anti Aging Cream Brazil

| | conc. % |
|---|---|
| INCI (EU) | by weight |
| Aqua | 67.15 |
| Glycerin; Aqua | 3.00 |
| Glycerin; Aqua; Sodium Levulinate; Sodium Anisate | 3.5 |
| Sodium Phytate; Aqua; Alcohol | 0.10 |
| Desferrioxamine | 0.10 |
| Xanthan Gum | 0.30 |
| Glyceryl Stearate Citrate; Cetearyl Alcohol; | 6.00 |
| Glyceryl Caprylate | |
| Glyceryl Caprylate | 0.2 |
| Carthamus tinctorius seed oil | 3.0 |
| Orbignya Oleifera Seed Oil | 4.0 |
| Limnanthes alba (seed) oil | 4.00 |
| Isoamyl Laurate | 6.00 |
| Tocopherol; Helianthus Annuus Seed Oil | 0.2 |
| Tocopherol; Helianthus Annuus Seed Oil | 0.20 |
| Parfum | 0.25 |
| Aqua; Sodium PCA | 2.00 |

### Natural Face Moisturizer

| | conc. % |
|---|---|
| INCI (EU) | by weight |
| Aqua | 66.12 |
| Glycerin | 3.00 |
| Desferrioxamine | 0.30 |
| Saccharide Isomerate; Aqua; Citric Acid; | 1.50 |
| Sodium Citrate | |
| Sodium Phytate; Aqua; Alcohol | 0.10 |
| Betaine | 2.00 |
| Sodium Anisate | 0.28 |
| Calcium Ketogluconate | 0.50 |
| Sodium Hyaluronate | 0.10 |
| Xanthan Gum | 0.15 |
| Distarch Phosphate | 1.00 |
| Sclerotium Gum | 0.15 |
| Glyceryl Stearate Citrate | 2.00 |
| Polyglyceryl-3 Stearate | 2.00 |
| Glyceryl Caprylate | 0.30 |
| Cetearyl Alcohol | 3.00 |
| Lavandula angustifolia oil | 2.00 |
| Isoamyl Laurate | 5.00 |
| Borago Officinalis Seed Oil | 1.00 |
| Macadamia Ternifolia Seed Oil | 2.00 |
| Virola Sebifera Nut Butter | 1.00 |
| Tocopherol; Helianthus Annuus Seed Oil | 0.50 |
| Glycerin; Aqua; Opuntia Ficus-Indica Stem Extract | 2.00 |
| Levulinic Acid; Sodium Levulinate; Aqua; Glycerin | 0.70 |
| Alcohol Denat. | 3.00 |
| Alcohol Denat.; Parfum | 0.30 |

### Natural Anti-Aging Cream

| | conc. % |
|---|---|
| INCI (EU) | by weight |
| Aqua | 56.25 |
| Desferrioxamine | 0.10 |
| Sodium Phytate; Aqua; Alcohol | 0.10 |
| Glycerin | 3.00 |
| Saccharide Isomerate; Aqua; Citric Acid; Sodium Citrate | 2.00 |
| Xanthan Gum | 0.25 |
| Distarch Phosphate | 1.00 |
| Carrageenan | 0.20 |
| Polyglyceryl-3 Stearate; Behenyl Alcohol; Glyceryl | 7.00 |
| Stearate Citrate; Helianthus Annuus Seed Cera; | |
| Glyceryl Caprate; Ascorbyl Palmitate; Tocopherol; | |
| Helianthus Annuus Seed Oil | |
| Behenyl Alcohol | 1.00 |
| Hydrogenated Vegetable Glycerides | 0.50 |
| Lauryl Olivate | 3.00 |
| Platonia Insignis Seed Butter | 2.00 |
| Isoamyl Laurate | 5.00 |
| Simmondsia Chinensis Seed Oil | 3.00 |
| Bertholletia Excelsa Seed Oil | 2.00 |
| Vitis Vinifera Seed Oil | 4.00 |
| Tocopherol; Helianthus Annuus Seed Oil | 0.30 |
| Maltodextrin; Aqua; Coenochloris Signiensis | 2.00 |
| Extract; Lecithin | |
| Aqua | 4.00 |
| Triethyl Citrate; Glyceryl Caprylate; Benzoic Acid | 1.00 |
| Glycerin; Aqua; Glutamine; Decyl Glucoside; Phenethyl | 2.00 |
| Alcohol; Citric Acid; Cistus Incanus Flower/Leaf/ | |
| Stem Extract; Gynostemma Pentaphyllum Leaf/Stem Extract | |
| Parfum | 0.30 |

### Natural Day Protection Face Cream

| | conc. % |
|---|---|
| INCI (EU) | by weight |
| Aqua | 60.25 |
| Glycerin | 4.00 |
| Desferrioxamine | 0.50 |
| Sodium Phytate; Aqua; Alcohol | 0.10 |
| Xanthan Gum | 0.75 |
| Distarch Phosphate | 1.00 |
| Polyglyceryl-3 Stearate; Behenyl Alcohol; Glyceryl | 6.00 |
| Stearate Citrate; Helianthus Annuus Seed Cera; | |
| Glyceryl Caprate; Ascorbyl Palmitate; Tocopherol; | |
| Helianthus Annuus Seed Oil | |
| Prunus Amygdalus Dulcis Oil | 5.00 |
| Helianthus Annuus Seed Oil | 3.00 |
| Oenothera Biennis Oil | 2.00 |
| Shorea Stenoptera Seed Butter | 3.00 |
| Isoamyl Laurate | 7.00 |
| Tocopheryl Acetate | 1.00 |
| Tocopherol; Helianthus Annuus Seed Oil | 0.20 |
| Glycerin; Aqua; Sodium Levulinate; Sodium Anisate | 3.00 |
| Octyldodecanol; Ribes Nigrum Seed Oil; Helianthus | 1.00 |
| Annuus Seed Oil Unsaponifiables; Cardiospermum | |
| Halicacabum Flower/Leaf/Vine Extract; Tocopherol; | |
| Helianthus Annuus Seed Oil; Rosmarinus Officinalis | |
| Leaf Extract | |
| Solanum Lycopersicum Fruit Extract; Helianthus Annuus | 2.00 |
| Hybrid Oil | |
| Parfum | 0.20 |

### Natural Night Cream

| | conc. % |
|---|---|
| INCI (EU) | by weight |
| Aqua | 62.60 |
| Glycerin | 7.00 |
| Desferrioxamine | 0.20 |
| Zinc Sulfate | 1.00 |
| Ectoin | 0.30 |
| Polyglyceryl-3 Polyricinoleate; Sorbitan Sesquioleate; | 8.50 |
| Cetyl Ricinoleate; Glyceryl Caprate; Cera Alba; | |
| Magnesium Stearate; Aluminum Tristearate | |
| Argana Spinosa Kernel oil | 8.00 |
| Squalane | 4.00 |
| Isoamyl Laurate | 6.00 |
| Passiflora Edulis Seed Oil; Oryza Sativa Bran Oil; | 2.00 |
| Euterpe Oleracea Fruit Oil; Orbignya Oleifera | |
| Seed Oil; Tocopherol; Helianthus Annuus Seed Oil | |
| Tocopherol; Helianthus Annuus Seed Oil | 0.20 |
| Parfum | 0.20 |

### Natural Night WO Cream

| | conc. % |
|---|---|
| INCI (EU) | by weight |
| Aqua | 62.60 |
| Glycerin | 7.00 |
| Desferrioxamine | 0.20 |
| Zinc Sulfate | 1.00 |
| Ectoin | 0.60 |
| Polyglyceryl-3 Polyricinoleate; Sorbitan Sesquioleate; | 8.50 |
| Cetyl Ricinoleate; Glyceryl Caprate; Cera Alba; | |
| Magnesium Stearate | |
| Persea Gratissima Oil | 8.00 |
| Squalane | 4.00 |
| Isoamyl Laurate | 6.00 |
| Passiflora Edulis Seed Oil; Oryza Sativa Bran Oil; | 2.00 |
| Euterpe Oleracea Fruit Oil; Orbignya Oleifera | |
| Seed Oil; Tocopherol; Helianthus Annuus Seed Oil | |
| Tocopherol; Helianthus Annuus Seed Oil | 0.20 |
| Parfum | 0.20 |

### Natural Foot Cream

| | conc. % |
|---|---|
| INCI (EU) | by weight |
| Aqua | 66.80 |
| Sodium Phytate; Aqua; Alcohol | 0.10 |
| Desferrioxamine | 0.10 |
| Glycerin | 3.00 |
| Aqua; Glycerin; Sodium Anisate | 2.00 |
| Xanthan Gum | 0.30 |
| Galactoarabinan | 0.20 |
| Glyceryl Stearate Citrate; Cetearyl Alcohol; | 5.00 |
| Glyceryl Caprylate | |
| Sodium Caproyl/Lauroyl Lactylate | 1.00 |
| Tocopherol; Helianthus Annuus Seed Oil | 0.20 |
| Cetearyl Alcohol | 3.00 |
| Olea Europaea Fruit Oil | 3.00 |
| Cera Alba | 1.00 |
| Virola Surinamensis Seed Butter | 2.00 |
| Rosa Canina Fruit Oil | 2.00 |
| Isoamyl Laurate | 3.00 |
| Triethyl Citrate | 3.00 |
| Menthyl Lactate | 0.30 |
| Aqua; Phospholipids; Stearic Acid; Palmitic Acid | 4.00 |

### Natural Repair Hand Cream

| | conc. % |
|---|---|
| INCI (EU) | by weight |
| Aqua | 67.35 |
| Glycerin | 3.50 |
| Desferrioxamine | 0.20 |
| Sodium Phytate; Aqua; Alcohol | 0.10 |
| P-Anisic Acid | 0.20 |
| Aqua; Sodium Hydroxide | 0.30 |
| Galactoarabinan | 0.30 |
| Xanthan Gum | 0.25 |
| Glyceryl Stearate Citrate; Cetearyl Alcohol; | 5.00 |
| Glyceryl Caprylate | |
| Behenyl Alcohol | 2.00 |
| Glyceryl Caprylate | 0.30 |
| Isoamyl Laurate | 8.00 |
| Garcinia Indica Seed Butter | 1.00 |
| Castor oil | 2.00 |
| Copaifera Officinalis Resin | 1.00 |
| Cocos Nucifera Oil | 4.00 |
| Palm oil | 2.00 |
| Glycine soja oil | 2.00 |
| Tocopherol; Helianthus Annuus Seed Oil | 0.50 |

### Natural Warming Foot Cream

| | conc. % |
|---|---|
| INCI (EU) | by weight |
| Water | 61.05 |
| Glycerin | 5.00 |
| Desferrioxamine | 0.10 |
| Sodium Phytate; Aqua; Alcohol | 0.10 |
| Glycerin; Aqua; Sodium Levulinate; Sodium Anisate | 3.50 |
| Magnesium Aluminum Silicate | 0.50 |
| Sclerotium Gum | 0.30 |
| Glyceryl Stearate Citrate | 0.50 |
| Polyglyceryl-3 Stearate | 4.00 |
| Virola Surinamensis Seed Butter | 5.00 |
| Citrus Medica Limonum Peel Oil | 3.00 |
| Sodium Caproyl/Lauroyl Lactylate | 0.50 |
| Glyceryl Stearate | 3.00 |
| Isoamyl Laurate | 8.00 |
| Melaleuca Alternifolia Leaf Oil | 5.00 |
| Tocopherol; Helianthus Annuus Seed Oil | 0.20 |
| Parfum | 0.25 |

1. A composition comprising
A) at least one substance selected from the group consisting of a desferrioxamine and an acid addition product of the desferrioxamine or a salt thereof,
B) a mixture comprising at least two triglycerides,
wherein the mixture of the at least two triglycerides comprised in the composition has an iodine value of from 6 cg I/g to 300 cg I/g,
wherein component B) is present in an amount of 5.0% by weight to 80% by weight, wherein the percentages by weight being with respect to the total composition.

2. The composition according to claim 1, wherein the desferrioxamine is selected from the group consisting of desferrioxamine A_{1A}, desferrioxamine A_{1B}, desferrioxamine A₂, desferrioxamine B, desferrioxamine D₁, desferrioxamine D₂, desferrioxamine E, desferrioxamine Et₁, desferrioxamine Et₂, desferrioxamine Et₃, desferrioxamine G₁, desferrioxamine G_{2A}, desferrioxamine G₂₃, desferrioxamine G_{2C}, desferrioxamine H, desferrioxamine N, desferrioxamine P₁, desferrioxamine T₁, desferrioxamine T₂, desferrioxamine T₃, desferrioxamine T₇, desferrioxamine T₈, desferrioxamine Te₁, desferrioxamine Te₂, desferrioxamine Te₃, desferrioxamine X₁, desferrioxamine X₂, desferrioxamine X₃ and desferrioxamine X₄.

3. The composition according to claim 1, wherein the acid addition product of the desferrioxamine or the salt thereof is selected from the group consisting of the acid addition product of formic acid, aminooxyacetic acid, amino acids, anthraquinonecarboxylic acid, succinic acid, aqueous hydrochloric acid, acetic acid, folic acid, glutaric acid, hydroxamic acid, malonic acid, methanesulfonic acid, nalidixic acid, N-(methylamino)benzoic acid, phenylsuccinic acid, phenylglutaric acid, phosphorous acid, phosphoric acid, sulfurous acid, sulfuric acid, tartaric acid and citric acid.

4. The composition according to claim 1, wherein component A) is present in an amount of 0.005% by weight to 10.0% by weight, wherein the percentages by weight being with respect to the total composition.

5. The composition according to claim 1, wherein the mixture comprised in component B) comprises at least three triglycerides.

6. The composition according to claim 1 wherein the mixture comprised in component B) is obtained from a natural source.

7. The composition according to claim 1, wherein the mixture comprised in component B) is selected from the group consisting of a natural oil, a natural wax, a natural butter, and an essential oil or a mixture thereof.

8. The composition according to claim 1, wherein the composition is an emulsion.

9. The composition according to claim 1, wherein at least one further additional component is selected from the group consisting of an emollient, an emulsifier, a coemulsifier, a thickener/viscosity regulator/stabilizer, an antioxidant, a hydrotrope(or polyol), a solid and filler, a pearlescent additive, an insect repellent, a self-tanning agent, a preservative, a conditioner, a perfume, a colourant, a cosmetic active substance, a care additive, a superfatting agent and a solvent.

10. A method for preventing autoxidation of a mixture comprising at least two triglycerides, the method comprising:
applying at least one substance selected from the group consisting of a desferrioxamine and an acid addition product of the desferrioxamine or a salt thereof to a mixture, wherein the mixture of the at least two triglycerides has an iodine value of from 6 cg I/g to 300 cg I/g.

11. A method for decreasing and/or preventing formation of malodour in a composition, the method comprising:
applying at least one desferrioxamine and/or at least one acid addition product of a desferrioxamine or a salt thereof to a composition, wherein the composition comprises a mixture of at least two triglycerides.

12. A method for decreasing and/or preventing formation of toxicological-relevant oxidation degradation product in a composition comprising:
applying at least one desferrioxamine and/or at least one acid addition product of a desferrioxamine or a salt thereof to a composition, wherein the composition comprises a mixture of at least two triglycerides having an iodine value of from 6 cg I/g to 300 cg I/g.
